(19) United States
(12) Patent Application Publication
(10) Pub. No.: US 2024/0297015 A1
(43) Pub. Date: Sep. 5, 2024

(54) METHOD OF PREPARING A SAMPLE FOR TRANSMISSION ELECTRON MICROSCOPY (TEM) ANALYSIS

(71) Applicant: FEI Company, Hillsboro, OR (US)

(72) Inventors: Ludek Cervinka, Slavkov u Brna (CZ); Jaroslav Starek, Krelov-Bruchotin (CZ); Tomas Onderlicka, Brno (CZ); Lukas Hubner, Jirikovice (CZ)

(21) Appl. No.: 18/178,156

(22) Filed: Mar. 3, 2023

(51) Int. Cl. H01J 37/30; G01N 33/483; H01J 37/305

(52) U.S. Cl. CPC H01J 37/3005; G01N 33/4833; H01J 37/305; H01J 2237/2802; H01J 2237/2803

(57) ABSTRACT

A method of preparing a sample for transmission electron microscopy (TEM) analysis is provided. The method comprises cleaning the sample to remove a redeposition layer, imaging the cleaned sample and identifying a location of a region of interest within the sample, and removing material from the sample, based on the identified location of the region of interest within the sample. Advantageously, the sample thinning step is performed based on a detected location of a region of interest. This thinning step involves removal of uneven surfaces (the “lamella roof”) and thinning the remaining bulk substrate to remove redundant material, so that the silicon substrate volume between the surface of the sample and the region of interest has a defined thickness.

### TECHNICAL FIELD

The present invention relates to automated methods for preparing TEM samples from semiconductor devices.

### BACKGROUND

Transmission electron microscopes (TEMs) are used for monitoring semiconductor manufacturing processes, analyzing defects, and investigating interface layer morphology. TEMs allow observation of features having sizes on the order of nanometers. In contrast to SEMs, which only image the surface of a material, TEMs also allows analysis of the internal structure of a sample. In a TEM, a broad beam impacts the sample and electrons that are transmitted through the sample are focused to form an image of the sample. The sample must be sufficiently thin to allow many of the electrons in the primary beam to travel though the sample and exit on the opposite site. Because a sample must be very thin for viewing with transmission electron microscopy, preparation of the sample can be delicate and time-consuming.

Some techniques for preparing TEM samples may involve cleaving, chemical polishing, mechanical polishing, or broad beam low energy ion milling. Combinations of these techniques are also possible. These techniques often require that the starting material be sectioned into smaller and smaller pieces, thereby destroying much of the original sample.

Other techniques generally referred to as “lift-out” procedures use a focused ion beam (FIB) to cut the sample from a substrate while greatly limiting or eliminating damage to surrounding areas of the substrate. These techniques are useful for analyzing the results of semiconductor manufacture, for example.

A sample is typically prepared from a larger bulk sample by milling away material with an ion beam to create trenches on either side of the region of interest, leaving a thin section referred to as a “lamella.” The lamella is partly severed from the sample substrate by ion beam milling around the bottom and the sides of the lamella until it is connected to the substrate only by a small amount of material.

The process of creating and extracting a lamella and transferring it to the sample grid is a delicate and time-consuming procedure, often requiring about 45 to 90 minutes per sample, and requiring the constant operator attention of a skilled operator. For total analysis of an area of interest on a semiconductor wafer, it may be desirable to analyze as many as, for example, 15 to 50 or more TEM samples. When so many samples must be extracted and measured, the total time to process the samples from one area can be hours or even days. Thus, even though the information that can be gained through TEM analysis can be very valuable, the process has been prohibitively time consuming for manufacturing process control and other routine procedures.

Improving the speed at which lamellae can be prepared for imaging therefore would provide significant advantages in both time and potential revenue by allowing work pieces selected for analysis to return to the production line more quickly. Automation of the lamella preparation process would not only speed up the process but also reduce the level of expertise required for operators, representing an advantage for the manufacturer. In addition, a skilled operator may be performing other tasks while automatic operations are being performed, increasing the throughput of the procedure.

Due to the precision required to mill, extract, transfer, and deposit a lamella on a sample grid, the process has not adapted itself to automation. As the lamella thickness is reduced, it becomes more likely that the region of interest will be excluded from the lamella. Lamellae under 100 nm in thickness, particularly lamellae under 70 nm, are difficult to produce either manually or automatically.

As lamellae become thinner, they can warp due to thermal or mechanical stress, changing their positions relative to the beam, which can ruin the lamella by allowing the beam to impact the region of interest. Thickness variations in the lamella can result in sample bending, over-milling, or other catastrophic defects that render the sample useless. In addition, the sample probe for manipulation of the lamella must be placed with extreme precision when preparing to extract the lamella from the substrate, and also when landing the lamella on the sample grid. These factors combine to make the preparation of lamella for analysis an exceedingly difficult process to automate.

US2019272975A1 describes automation of the sample preparation procedure. Some involve the use of machine vision to determine the positions of the lamella, the probe, and/or the TEM grid to guide the attachment of the probe to the lamella and the attachment of the lamella to the TEM grid. In some examples, reference structures known as “fiducials,” formed by a charged particle beam on the lamella and/or the probe, can be used to guide the tip to the vicinity of the lamella, and to guide the attachment of the probe to the lamella and the attachment of the lamella to the TEM grid. A fiducial may be formed by milling a structure into the surface or by depositing material onto the surface. However, there still exists a problem of thinning the TEM sample so that the region of interest may be properly imaged.

TEM samples are typically less than 100 nm thick, but for some applications samples must be considerably thinner. With advanced processes at 30 nm and below, the sample needs to be less than 20 nm in thickness in order to avoid overlap among small scale structures. Currently thinning below 30 nm is difficult and not robust. Thickness variations in the sample result in sample bending, overmilling, or other catastrophic defects. For such small samples, preparation is a critical step in TEM analysis that significantly determines the quality of structural characterization and analysis of the smallest and most critical structures.

A significant problem for the preparation of ultra thin (<30 nm thick) TEM samples is commonly referred to as “curtaining,” in which non-uniform high-density materials on the surface of an integrated circuit produce a non-planar face on the TEM sample after thinning. Top-down thinning of a sample having these types of structural or density variations will cause vertical ridges to propagate from the denser materials (i.e. metal lines) near the top of the sample (the top being defined as closest to the ion beam source) down the face of the cross-section, running in a direction parallel to the ion beam direction. Curtaining is most often observed in semiconductor materials where multiple patterned layers of materials having a low sputtering yield blocks a faster sputtering yield material. Curtaining may also be observed in materials exhibiting different topographic regions where changes in sputtering yields vary with the milling incident angle. Curtaining artifacts reduce the quality of the TEM imaging and limit the minimal useful specimen thickness. For thin TEM samples having a thickness of less than 30 nm, the two cross-section faces are in very close proximity so thickness variations from curtaining effects can cause a sample to be unusable.

In order to minimize curtaining in TEM sample preparation, it is known to invert the samples so that the bottom of the sample (the substrate) is facing the FIB column. Because the substrate portion of the sample will not have imbedded features such as metal lines or transistors, curtaining artifacts will not be introduced into the portion of the sample face containing the region of interest, i.e., the layers of circuitry on the top surface of the semiconductor. While this technique works reasonably well for TEM samples having a thickness of 50 to 100 nm, for ultra-thin samples having a sample thickness of 30 nm or less, even samples prepared by inverting the sample before thinning often show milling artifacts resulting in a undesirably non-uniform sample face.

WO2012103534 describes methods of preparing ultra-thin TEM samples by combining inverted thinning with an additional cleaning step to remove surface defects on the FIB-facing substrate surface. WO2012103534 also describes that the FIB can be used to mill away the “dirty” substrate surface, forming a cleaned, uniformly flat substrate surface that functions as a sort of “hard mask” during TEM sample thinning in that it protects the region of interest below (when the sample is inverted) and it controls the creation of a smooth, flat TEM sample face. However, thinning the TEM sample to the correct depth is still time consuming and requires intervention from a skilled operator.

### SUMMARY

The present invention provides a method of preparing a sample for transmission electron microscopy (TEM) analysis. The method comprises:
cleaning the sample to remove a redeposition layer;
imaging the cleaned sample and identifying a location of a region of interest within the sample; and
removing material from the sample, based on the identified location of the region of interest within the sample.

Advantageously, the sample thinning step is performed based on a detected location of a region of interest. This thinning step involves removal of uneven surfaces (the “lamella roof”) and thinning the remaining bulk substrate to remove redundant material, so that the silicon substrate volume between the surface of the sample and the region of interest has a defined thickness.

The sample is cleaned to remove the redeposition layer. This may be performed after the lamella has been extracted from the bulk substrate. Alternatively, this may be performed sooner in the workflow. For example, the cleaning step can be performed during or prior to the liftout of the lamella from the bulk sample.

Removing material from the sample may comprise:
removing a first portion of the sample using an ion beam oriented in a first direction relative to the sample; and
removing a second portion of the sample using an ion beam oriented in a second direction relative to the sample, where the second direction is perpendicular to the first direction.

Removing material from the sample may comprise ion beam milling with a focussed ion beam (FIB).

The FIB in the cleaning step may be perpendicular to the thinning direction and parallel to the CLR direction.

As described above, the roof of the lamella (the first portion) is removed (or “clipped”), so that the lower face of the lamella is flat and perpendicular to the FIB, in preparation for the thinning step (during which the second portion is removed). The lower face of the lamella may therefore be flat and perpendicular to the FIB during the thinning step.

Material may be removed during the clipping step by ion milling using a FIB that is perpendicular to the thinning direction. The “clipping lamella roof” CLR step (during which the first portion is removed) may be performed as a preparation step in advance of the thinning step (during which the second portion is removed). The thinning step may be performed using an FIB in the thinning direction. Where the FIB directions between the CLR step and the thinning step are described as being “perpendicular”, this may mean that the same FIB is used and that the lamella is rotated through 90 degrees between operations.

Cleaning the sample to remove a redeposition layer may comprise removing the redeposition layer from a first face of the sample. The first face may be perpendicular to the first direction.

Removing a first portion of the sample exposes a second face of the sample perpendicular to the first face of the sample. The second face may be perpendicular to the second direction.

The second face may be perpendicular to the thinning direction (perpendicular to the FIB during removal of the second portion).

The first portion of the sample may comprise a surface of the sample that is angled and/or non-planar.

Where the surface is described as “angled”, this may mean that the surface is not perpendicular to the first face, not parallel to the second face and/or not perpendicular to the FIB in the thinning direction (the second direction).

Where the surface is described as “non-planar”, this may mean that the surface is not flat. For example, the surface may comprise balls and bumps on the face (e.g., due to liftout of the lamella from the bulk substrate).

Removing the first portion of the sample may comprise ion milling a portion of the first face of the sample all the way through the sample to expose the second face. Following removal of the first portion, the second face of the lamella may be flat and perpendicular to the second direction.

Thinning the sample may comprise milling the sample to remove the balls and bumps so that the second face becomes flat. Thinning the sample may comprise milling the sample to remove the angled surface resulting from the bulk milling (roof shape).

Removing the second portion of the sample may comprise removing material from the second face of the sample. Material may be removed from the second face of the sample via ion milling with the FIB in the thinning direction.

Removing material from the second face of the sample may comprise:
applying a fiducial to the second face;
ion milling the second face;
imaging the lamella; and
controlling the ion milling to correct for drift, based on an imaged location of the fiducial.

Alternatively, removing material from the second face of the sample may comprise:
referencing a fiducial adjacent to the second face, where the fiducial is located on a sample grid carrying the sample;
ion milling the second face;
imaging the lamella; and
controlling the ion milling to correct for drift, based on an imaged location of the fiducial.

Removing material from the second face of the sample may be based on the identified location of the region of interest within the sample, so that material is removed until a predefined thickness of material remains between the second face of the sample and the region of interest.

The method may further comprise repositioning the sample (e.g., by a 90 degree rotation) between removing the first portion of the sample and removing the second portion of the sample. In this way, the same ion beam is used with the sample orientation changing, rather than having separate ion beams.

Cleaning, imaging and clipping steps may be performed with the sample at the same position. Alternatively, the sample may be repositioned during removing, cleaning, imaging, and/or clipping.

After removal of the sample from the bulk substrate, the probe may be driven to position the sample for the cleaning or imaging step (depending on whether the cleaning step is performed before, during or after removal of the lamella). The orientation of the sample during removal is usually different to the orientation of the sample during imaging.

After the clipping step the sample may be moved to align it for the thinning step. However, this is not mandatory (a perpendicular FIB may be used to eliminate the need for sample rotation between these steps).

The CLR step may be performed with the FIB perpendicular to the thinning direction, as the CLR prepares the lamella for the thinning by smoothing and flattening the second surface.

Cleaning the sample to remove a redeposition layer may comprise removing the redeposition layer from a first face of the sample. Imaging the cleaned sample may comprise using a charged particle beam to provide an image of the first face of the sample.

The charged particle beam may be an electron beam or an ion beam.

The image may be provided by a scanning electron microscope (SEM) imaging process. Alternatively, the image may be obtained via FIB imaging.

The sample may be a lamella, and the method may further comprise separating the lamella from a bulk substrate.

An angled and bumpy surface may be exposed when the lamella is removed from the bulk substrate. The above methods are particularly useful for dealing with this situation.

The method may further comprise thinning the sample by an inverted lamella thinning process. Therefore, the surface to be subjected to the thinning step is the uneven surface created when the lamella is removed from the bulk.

The inverted lamella thinning process may comprise applying a fiducial to the lamella, imaging the lamella and controlling the inverted lamella thinning process to correct for drift, based on an imaged location of the fiducial.

Separating the lamella from the bulk substrate may comprise ion beam milling.

Cleaning the sample to remove the redeposition layer may be performed during or prior to separating the lamella from the bulk substrate (“liftout”).

Identifying a location of a region of interest within the sample may comprise performing image processing techniques to identify an interface between a substrate layer and a structure layer of the sample

In a first example, the region of interest may be a layer of transistors. The interface between the substrate and the transistors layer may define the start of the region of interest and may be detectable using image processing techniques. For different samples, different structures may define the region of interest (not just substrate/transistors interface). In general unique features within the sample may define the region of interest.

Identifying the location of the region of interest may comprise performing image processing techniques via segmentation using a convolutional neural network (CNN) to reduce a detection area and using image processing methods (such as “classic” methods) to identify the interface.

The present invention also provides an apparatus configured to perform a method described above. The apparatus may be an apparatus for preparing a TEM sample, which may comprise:
an ion beam system including an ion beam source, optics for focusing an ion beam along an axis and onto a substrate, and a micromanipulator for manipulating the sample; and
a computer-readable memory storing computer software comprising instructions for controlling the apparatus and causing the apparatus to carry out a method described above.

The present invention also provides computer software comprising instructions that, when executed by a processor of an apparatus for preparing a TEM sample, cause the apparatus to perform a method described above.

### BRIEF DESCRIPTION OF THE DRAWINGS

FIG. 1A illustrates separation of a lamella from the bulk substrate and illustrates the lamella roof.

FIG. 1B illustrates a non-inverted thinning method.

FIG. 1C illustrates clipping a lamella roof in an inverted thinning method

FIG. 2A illustrates imaging a thinning fiducial from different directions during a thinning process.

FIG. 2B illustrates a lamella before thinning with thinning fiducial visible.

FIG. 2C illustrates an example in which the thinning fiducial is not visible in SEM, without first clipping the lamella roof.

FIG. 3A illustrates removal of the lamella roof with the FIB perpendicular to the thinning surface.

FIG. 3B illustrates perpendicular ion beam directions for the thinning step and the CLR step.

FIG. 4 illustrates layers of an example lamella.

FIG. 5 illustrates an example workflow for a “clip lamella roof” activity.

FIG. 6 illustrates lamella segmentation by a CNN.

FIG. 7A illustrates identification of a transistors layer.

FIG. 7B illustrates further transistors interface detection examples.

FIG. 8 illustrates identification of an upper position of the transistors interface.

FIG. 9 illustrates clipping redundant material in an example lamella.

FIG. 10 illustrates a dual beam FIB/STEM system that could be used to implement the invention.

### DETAILED DESCRIPTION

A sample is typically prepared from a larger bulk sample by milling away material with an ion beam to create trenches on either side of the region of interest, leaving a lamella. The lamella is partly severed from the sample substrate by ion beam milling around the bottom and the sides of the lamella until it is connected to the substrate only by a small amount of material. Steps for extracting a lamella from a bulk substrate are described in US2019272975A1, which is hereby incorporated in its entirety.

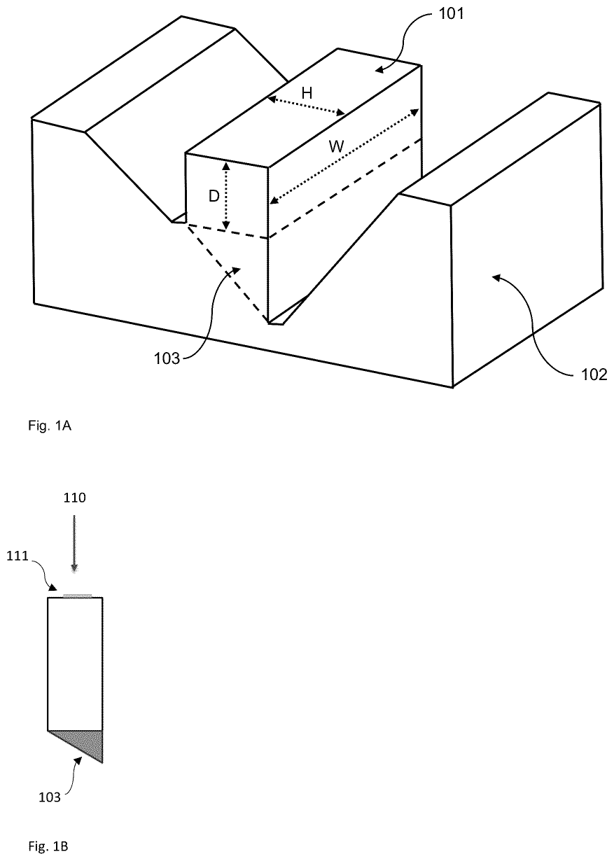

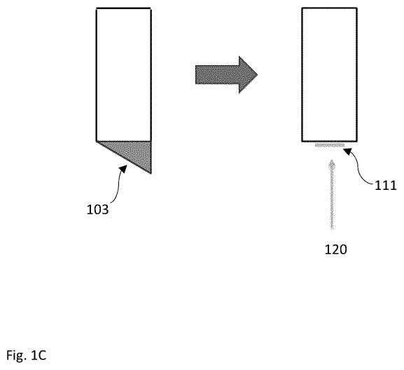

Formation of a lamella prior to removal from the bulk substrate is illustrated in FIG. 1A. The lamella 101, having dimensions H, W and D, is separated from the bulk substrate 102 by milling trenches on either side. The lamella 101 remains attached to the bulk substrate 102 by a small section of material 103. This section 103 will remain attached to the lamella when the lamella is lifted out and separated from the bulk substrate. Due to the way that the trenches are formed, the section of material 103 is angled with respect to the lamella and is referred to as a “lamella roof” 103.

In a non-inverted thinning method, as illustrated in FIG. 1B, ion beam milling is applied in the thinning direction 110, which is from the face of the lamella opposite the roof 103. A thinning fiducial 111 is etched on the face of the lamella to be thinned. Since the thinning direction 110 is opposite the lamella roof 103, removal of the lamella roof 103 is not required.

An inverted thinning method, as illustrated in FIG. 1C, is performed in a two-step process. First, the lamella roof 103 may be removed in a stage called “clipping lamella roof” or CLR. This step removes the angled surface of the lamella roof 103 and creates a perpendicular surface, on which a thinning fiducial 111 may be etched. Second, thinning of the lamella is performed in the inverted thinning direction 120. The inverted lamella thinning process comprises applying a fiducial to the lamella. During thinning, the lamella is imaged via SEM and the inverted lamella thinning process is controlled to correct for drift, based on an imaged location of the fiducial. The SEM electron beam may be angled relative to the FIB. In some examples, the SEM is tilted by 52 degrees From the FIB.

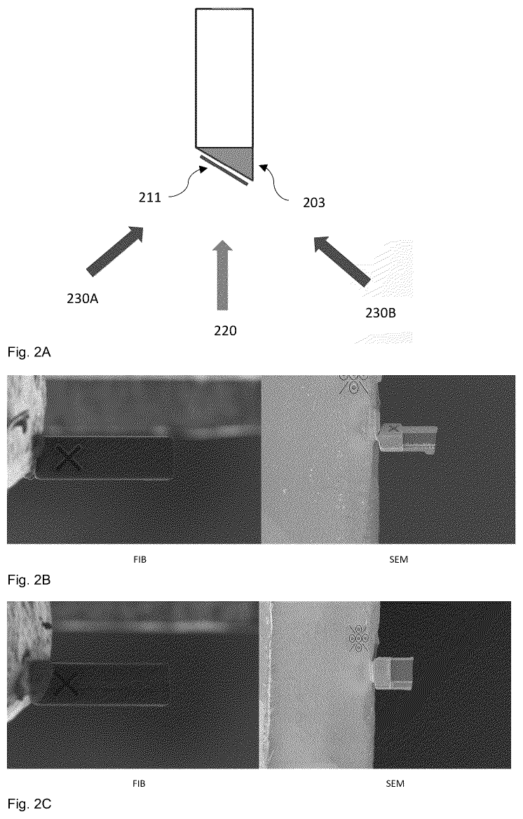

When extracting thick lamella from the bulk sample there is an angled face at the bottom. During the inverted lift-out this face is then transferred to the top of the thick lamella (hence why the angled portion is called “the roof”). This surface faces directly to the FIB during inverted thinning. If the angled surface were not removed, so that the surface on which the thinning is performed is perpendicular to the FIB, the robustness of the thinning process would be affected. This is illustrated in FIG. 2A, in which a fiducial 211 is applied to the angled face of the roof 203. The FIB direction 220 is therefore not perpendicular to the thinning surface on which the fiducial 211 is etched. The thinning fiducial is visible from the first SEM side 230A. However, from the second SEM side 230B, the thinning fiducial is obscured due to the angle of the lamella roof.

An example lamella before thinning is performed is illustrated in FIG. 2B, which illustrates an FIB image and a SEM image. The thinning fiducial is visible in both the FIB and the SEM images in FIG. 2B. However, in some cases, the roof angle presents a limitation to creating and using a thinning fiducial and a thinning protective layer, which has a direct impact on the robustness of the process. For example, in FIG. 2C, the thinning fiducial is not visible in the SEM image. This is due to the roof cut and uneven deposition thickness and shape resulting in the top of the lamella not being visible in the SEM image. Clipping the lamella roof so that the thinning surface is perpendicular to the FIB is therefore important for a robust automated thinning process.

The thinning process is continued until a predefined thickness of material remains between the region of interest (e.g., the transistor layer) and the surface of the lamella. Defining a silicon substrate above the region of interest is therefore an important step for the thinning process. The substrate serves as a protective caping on the region of interest and helps to prevent bending of the lamella. The exactly defined substrate volume has a direct impact on the repeatability and robustness of the thinning process.

During the sample extraction process, the bottom surface of the lamella will accumulate redeposited material from the sample extraction ion milling process. This material may be in the form of small balls and bumps. Non-uniformities on the bottom surface can also result from milling artifacts accrued during the bulk material removal process. These material or topographical variations on the surface of the substrate side of the sample have a significant effect upon the lamella thinning and could cause curtains later in the process. These types of surface variations propagate through the milling process as the lamella is thinned and result in sidewall non-uniformities that limit the minimum thickness to which the sample may be thinned. By removing the lamella roof prior to thinning, surface defects on the thinning surface of the lamella are removed and the resulting surface is smooth, flat and perpendicular to the thinning direction.

In other words, the FIB-facing substrate surface is “cleaned” with the FIB to form a uniform inverted substrate surface. The FIB can be used to mill away the “dirty” substrate surface, forming a cleaned, uniformly flat substrate surface that it protects the region of interest during thinning. The reliability of these methods make them especially suitable for automated sample preparation.

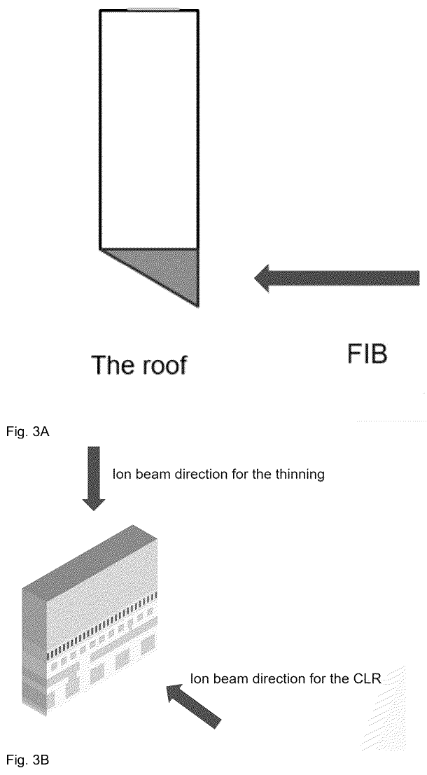

As illustrated in FIG. 3A, the lamella roof is removed with the FIB perpendicular to the thinning surface (the second face). As illustrated in FIG. 3B, the ion beam direction for the thinning step is perpendicular to the ion beam direction for the CLR step (the removal of the lamella roof).

For CLR, only part of the lamella is milled away. For example, the roof illustrated in FIG. 3A may be removed by placing the beam only over the section indicated and milling there long enough to remove all the material in the way of the beam.

Likewise, for the thinning step, only part of the lamella is milled away. However, for the thinning step, this may be achieved by scanning the beam over the second face and milling for a limited time, so that only a portion of the lamella is removed.

Steps of creating a thin TEM sample are described in WO2012103534, which is hereby incorporated by reference in its entirety.

An automated workflow for inverted thinning of a lamella consists of several steps. One important step is a clip lamella roof activity (CLR or CLRa), which prepares the lamella for thinning. The roof clipping facilitates fast and successful thinning. In order to provide an automated method, the roof clipping step may be fully automatic.

The main objectives are:
to find and define the position of the region of interest (such as the transistors layer);
to prepare surface for a thinning fiducial and a protective layer placement and remove redeposition; and
to set a volume of a silicon substrate up to the region of interest (such as the transistors layer).

A region of interest (such as a transistors layer) can be identified in order to define the extent of the thinning step. In order to identify the region of interest, an imaging step is performed. Prior to imaging the sample, a step of cleaning the sample to remove a redeposition layer is performed.

During the clip lamella roof activity, the lamella front face (the first face) is perpendicular to the beam. Therefore, imaging of the front face may be performed with the beam in the same orientation as the CLR activity (before, after or during the CLR activity).

The front face is the face where the structures of the device are visible. For CLR the front face is oriented so the FIB is perpendicular to the front face (the first face). After CLR the sample is reoriented so that the structures are not visible in FIB. In the new orientation, the newly exposed face from CLR is visible to FIB and the thinning step is performed in this orientation. The thinning step is controlled so that only a portion of the lamella is removed, to leave a predefined thickness of material between the thinning surface and the region of interest.

The region of interest is detected by using image processing methods. The found position is stored to guide the thinning step and to improve robustness and throughput of the process.

So that the structures can be effectively imaged, a redeposition layer must be removed from the front face to expose the structures. For this cleaning step, the whole lamella is scanned with the ion beam, but only for a limited time. Therefore, the ion beam is removing material from all over the exposed side of the lamella. However, since it is only for a short time, only a couple of tens of nm are removed. The direction of the FIB for the cleaning step is perpendicular to the front face of the lamella.

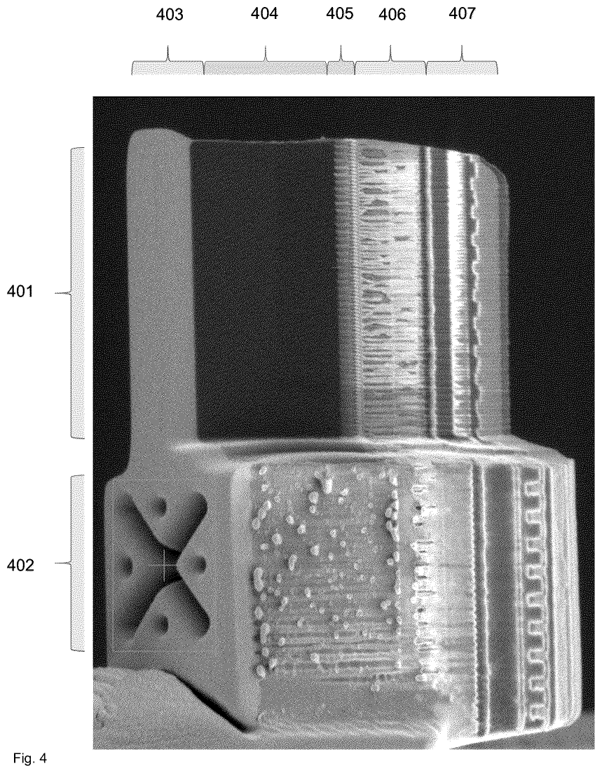

FIG. 4 illustrates an example lamella and identifies layers that may be present, including:
401: The thinning part of the lamella;
402: The thinning fiducial;
403: The protective layer;
404: The silicon substrate volume;
405: The transistors layer;
406: The capacitors layer; and
407: The rest of the layers.

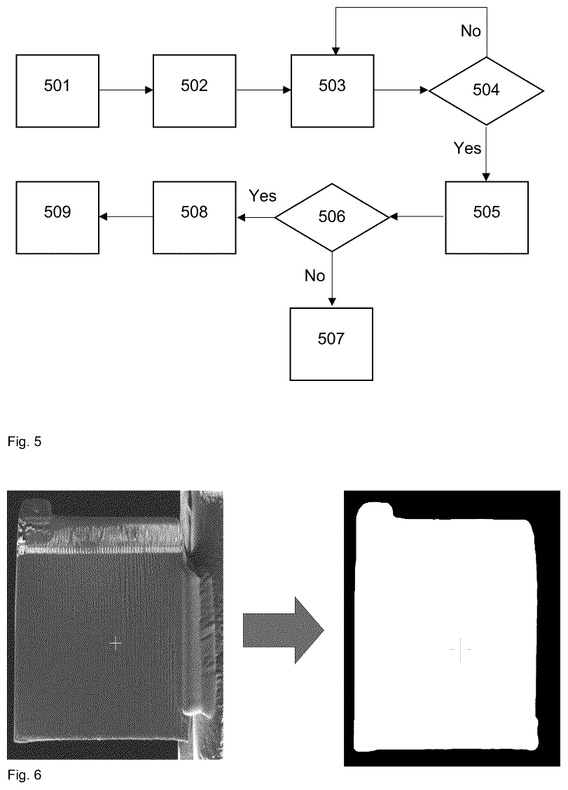

FIG. 5 illustrates a flowchart of an example workflow. At step 501, the lamella is successfully welded to a grid. At step 502, the stage is moved to a clipping position. A stage rotation may be used to position the lamella cut face perpendicular to the FIB beam. The lamella is centred in the correct position for surface cleaning. At step 503, a lamella's surface is cleaned. At step 504, the system determines whether the redeposition is removed. If no, the method returns to step 503. If yes, the method proceeds to step 505. This procedure is stopped when all redeposition (that was formed during lamella preparation and lift-out) is removed from the lamella surface or a maximum time of the cleaning is reached. At step 505, the transistors interface is detected. At step 506, the system determines whether the transistors are found. If no, the method proceeds to step 507. If yes, the method proceeds to step 508. At step 507, only the roof is clipped. If transistors are successfully detected using image processing methods then, based on the found position of the transistors layer, a clipping pattern is placed. At step 508, the lamella is clipped with a defined substrate volume. At step 509, a position of the transistors and a new lamella height is stored.

To automatically identify the region of interest (e.g., the transistors layer) is a challenging step. The lamella contains a few variables structures and it is desired to identify one of these automatically (e.g., the transistors layer). The proposed algorithm performs automatic identification in two stages.

First, rough lamella detection is performed using a convolution neural network (CNN). This produces a segmentation of the lamella to reduces the detection area and therefore reduce the time taken to perform the second stage. The result of lamella segmentation by this approach is shown in FIG. 6.

The second stage of the proposed algorithm is to find a characteristic layer (e.g. a transistors layer) from the segmented lamella image. The position of this layer strongly depends on the device from which the lamella is taken (memory or chip). The characteristic layer is detected without using a CNN or any previously stored information and the detection is made using “classic” image processing methods. Advantageously, this approach is suitable regardless of the kind of device that is being analysed and does not require re-training of the algorithm for new samples. The result is stored in the AutoTEM's internal parameter and in the image's metadata as well.

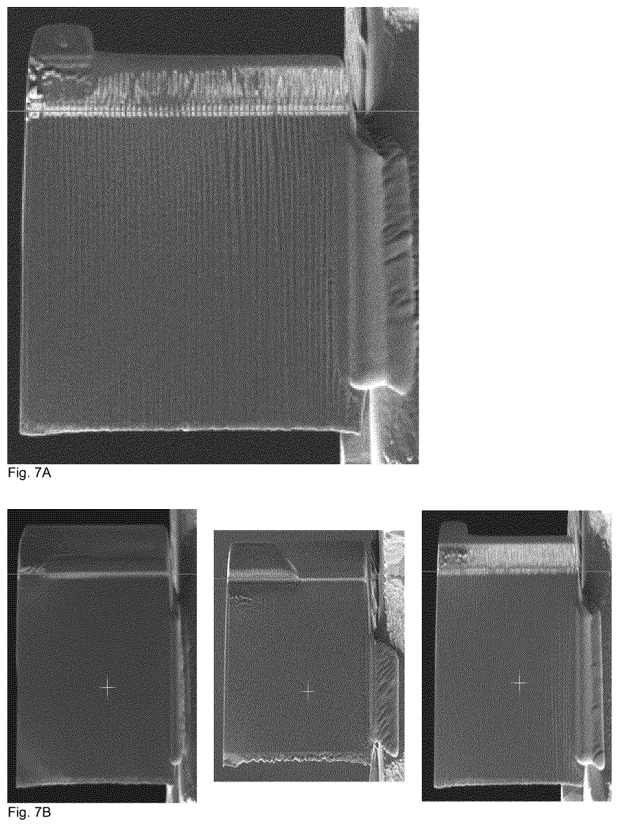

FIG. 7A illustrates identification of a transistors layer in an example sample. FIG. 7B illustrates further transistors interface detection examples.

In FIG. 8, the cross 801 shows an upper position of the transistors interface.

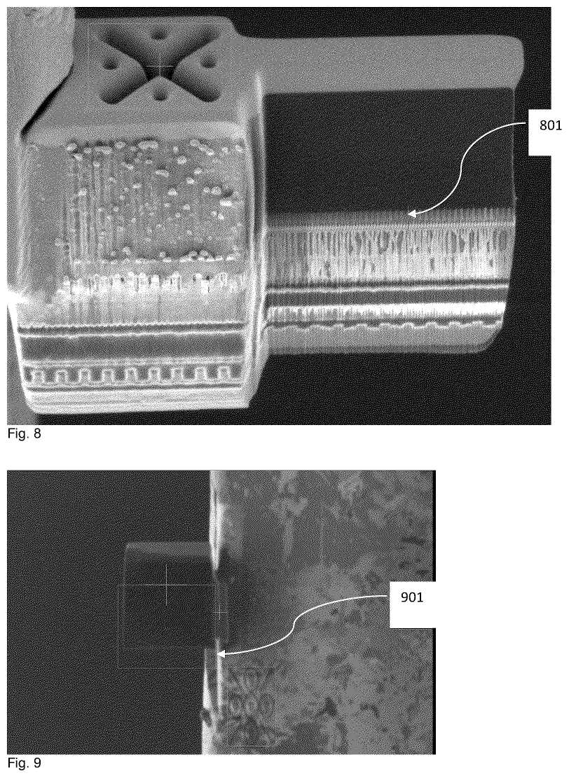

In FIG. 9, boxed region 901 illustrates redundant lamella material, which will be clipped in the CLR step. The size of the redundant material that is clipped in this step may be defined based on the location of the detected region of interest. The thinning step may also be controlled based on the detected location of the region of interest. Advantageously, a defined thickness of material may be retained adjacent to the region of interest. This region of material can provide a protective caping on the region of interest and can help to prevent bending of the lamella. The exactly defined substrate volume has a direct impact on the repeatability and robustness of the thinning process.

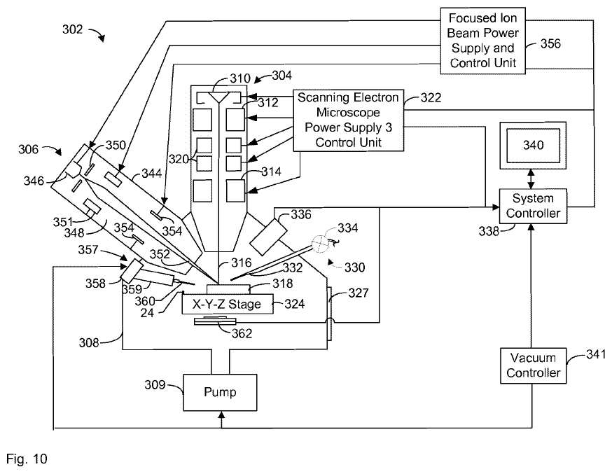

FIG. 10 depicts an exemplary dual beam SEM/FIB system 302 that can be used to carry out methods according to the present invention. Suitable dual beam systems are commercially available, for example, from FEI Company, Hillsboro, Oregon, the assignee of the present application. While an example of suitable hardware is provided below, the invention is not limited to being implemented in any particular type of hardware.

Dual beam system 302 has a vertically mounted electron beam column 304 and a focused ion beam (FIB) column 306 mounted at an angle of approximately 52 degrees From the vertical on an evacuable specimen chamber 308. The specimen chamber may be evacuated by pump system 309, which typically includes one or more, or a combination of, a turbo-molecular pump, oil diffusion pumps, ion getter pumps, scroll pumps, or other known pumping means.

The electron beam column 304 includes an electron source 310, such as a Schottky emitter or a cold field emitter, for producing electrons, and electron-optical lenses 312 and 314 forming a finely focused beam of electrons 316. Electron source 310 is typically maintained at an electrical potential of between 500 V and 30 kV above the electrical potential of a work piece 318, which is typically maintained at ground potential.

Thus, electrons impact the work piece 318 at landing energies of approximately 500 eV to 30 keV. A negative electrical potential can be applied to the work piece to reduce the landing energy of the electrons, which reduces the interaction volume of the electrons with the work piece surface, thereby reducing the size of the nucleation site. Work piece 318 may comprise, for example, a semiconductor device, microelectromechanical system (MEMS), or a lithography mask. The impact point of the beam of electrons 316 can be positioned on and scanned over the surface of a work piece 318 by means of deflection coils 320. Operation of lenses 312 and 314 and deflection coils 320 is controlled by scanning electron microscope power supply and control unit 322. Lenses and deflection unit may use electric fields, magnetic fields, or a combination thereof.

Work piece 318 is on movable stage 324 within specimen chamber 308. Stage 324 can preferably move in a horizontal plane (X and Y axes) and vertically (Z axis) and can tilt approximately sixty (60) degrees and rotate about the Z axis. A door 327 can be opened for inserting work piece 318 onto X-Y-Z stage 324 and also for servicing an internal gas supply reservoir (not shown), if one is used. The door is interlocked so that it cannot be opened if specimen chamber 308 is evacuated.

Mounted on the vacuum chamber are multiple gas injection systems (GIS) 330 (two shown). Each GIS comprises a reservoir (not shown) for holding the precursor or activation materials and a needle 332 for directing the gas to the surface of the work piece. Each GIS further comprises means 334 for regulating the supply of precursor material to the work piece. In this example the regulating means are depicted as an adjustable valve, but the regulating means could also comprise, for example, a regulated heater for heating the precursor material to control its vapor pressure.

When the electrons in the electron beam 316 strike work piece 318, secondary electrons, backscattered electrons, and Auger electrons are emitted and can be detected to form an image or to determine information about the work piece. Secondary electrons, for example, are detected by secondary electron detector 336, such as an Everhard-Thornley detector, or a semiconductor detector device capable of detecting low energy electrons.

STEM detector 362, located beneath the TEM sample holder 24 and the stage 324, can collect electrons that are transmitted through a sample mounted on the TEM sample holder. Signals from the detectors 336, 362 are provided to a system controller 338. Said controller 338 also controls the deflector signals, lenses, electron source, GIS, stage and pump, and other items of the instrument. Monitor 340 is used to display user controls and an image of the work piece using the signal

The chamber 308 is evacuated by pump system 309 under the control of vacuum controller 341. The vacuum system provides within chamber 308 a vacuum of approximately 3×10⁻⁶ mbar. When a suitable precursor or activator gas is introduced onto the sample surface, the chamber background pressure may rise, typically to about 5×10⁻⁵ mbar.

Focused ion beam column 306 comprises an upper neck portion 344 within which are located an ion source 346 and a focusing column 348 including extractor electrode 350 and an electrostatic optical system including an objective lens 351. Ion source 346 may comprise a liquid metal gallium ion source, a plasma ion source, a liquid metal alloy source, or any other type of ion source. The axis of focusing column 348 is tilted 52 degrees From the axis of the electron column. An ion beam 352 passes from ion source 346 through focusing column 348 and between electrostatic deflectors 354 toward work piece 318.

FIB power supply and control unit 356 provides an electrical potential at ion source 346. lon source 346 is typically maintained at an electrical potential of between 1 kV and 60 kV above the electrical potential of the work piece, which is typically maintained at ground potential. Thus, ions impact the work piece at landing energies of approximately 1 keV to 60 keV. FIB power supply and control unit 356 is coupled to deflection plates 354 which can cause the ion beam to trace out a corresponding pattern on the upper surface of work piece 318. In some systems, the deflection plates are placed before the final lens, as is well known in the art. Beam blanking electrodes (not shown) within ion beam focusing column 348 cause ion beam 352 to impact onto blanking aperture (not shown) instead of work piece 318 when a FIB power supply and control unit 356 applies a blanking voltage to the blanking electrode.

The ion source 346 typically provides a beam of singly charged positive gallium ions that can be focused into a sub one-tenth micrometer wide beam at work piece 318 for modifying the work piece 318 by ion milling, enhanced etch, material deposition, or for imaging the work piece 318.

A micromanipulator 357, such as the AutoProbe 200™ from Omniprobe, Inc., Dallas, Texas, or the Model MM3A from Kleindiek Nanotechnik, Reutlingen, Germany, can precisely move objects within the vacuum chamber. Micromanipulator 357 may comprise precision electric motors 358 positioned outside the vacuum chamber to provide X, Y, Z, and theta control of a portion 359 positioned within the vacuum chamber. The micromanipulator 357 can be fitted with different end effectors for manipulating small objects. In the embodiments described herein, the end effector is a thin probe 360. As is known in the prior art, a micromanipulator (or microprobe) can be used to transfer a TEM sample (which has been freed from a substrate, typically by an ion beam) to a TEM sample holder 361 for analysis.

System controller 338 controls the operations of the various parts of dual beam system 302. Through system controller 338, a user can cause ion beam 352 or electron beam 316 to be scanned in a desired manner through commands entered into a conventional user interface (not shown). Alternatively, system controller 338 may control dual beam system 302 in accordance with programmed instructions.

The term “TEM” as used herein refers to a transmission electron microscope or (TEM) or scanning transmission electron microscopy (STEM). References to preparing a sample for a TEM are to be understood to also include preparing a sample for viewing on a STEM. The term “STEM” as used herein also refers to both TEM and STEM.

Where the term “perpendicular” is used to describe angles between faces or beam directions, the skilled person will understand that the angle is 90 degrees but does not need to be exactly 90 degrees. There can be some small angle deviation.

Although the description of the present invention above is mainly directed at methods of preparing ultra thin TEM samples, it should be recognized that an apparatus performing the operation of such a method would further be within the scope of the present invention. Further, it should be recognized that embodiments of the present invention can be implemented via computer hardware, a combination of both hardware and software, or by computer instructions stored in a non-transitory computer-readable memory. The methods can be implemented in computer programs using standard programming techniques—including a non-transitory computer-readable storage medium configured with a computer program, where the storage medium so configured causes a computer to operate in a specific and predefined manner-according to the methods and figures described in this Specification. Each program may be implemented in a high level procedural or object oriented programming language to communicate with a computer system. However, the programs can be implemented in assembly or machine language, if desired. In any case, the language can be a compiled or interpreted language. Moreover, the program can run on dedicated integrated circuits programmed for that purpose.

Further, methodologies may be implemented in any type of computing platform, including but not limited to, personal computers, mini-computers, main-frames, workstations, networked or distributed computing environments, computer platforms separate, integral to, or in communication with charged particle tools or other imaging devices, and the like.

Aspects of the present invention may be implemented in machine readable code stored on a storage medium or device, whether removable or integral to the computing platform, such as a hard disc, optical read and/or write storage mediums, RAM, ROM, and the like, so that it is readable by a programmable computer, for configuring and operating the computer when the storage media or device is read by the computer to perform the procedures described herein. Moreover, machine-readable code, or portions thereof, may be transmitted over a wired or wireless network. The invention described herein includes these and other various types of computer-readable storage media when such media contain instructions or programs for implementing the steps described above in conjunction with a microprocessor or other data processor. The invention also includes the computer itself when programmed according to the methods and techniques described herein.

Computer programs can be applied to input data to perform the functions described herein and thereby transform the input data to generate output data. The output information is applied to one or more output devices such as a display monitor. In preferred embodiments of the present invention, the transformed data represents physical and tangible objects, including producing a particular visual depiction of the physical and tangible objects on a display.

Preferred embodiments of the present invention also make use of a particle beam apparatus, such as a FIB or SEM, in order to image a sample using a beam of particles. Such particles used to image a sample inherently interact with the sample resulting in some degree of physical transformation. Further, throughout the present specification, discussions utilizing terms such as “calculating,” “determining,” “measuring,” “generating,” “detecting,” “forming,” or the like, also refer to the action and processes of a computer system, or similar electronic device, that manipulates and transforms data represented as physical quantities within the computer system into other data similarly represented as physical quantities within the computer system or other information storage, transmission or display devices.

The invention has broad applicability and can provide many benefits as described and shown in the examples above. The embodiments will vary greatly depending upon the specific application, and not every embodiment will provide all of the benefits and meet all of the objectives that are achievable by the invention. Particle beam systems suitable for carrying out the present invention are commercially available, for example, from FEI Company, the assignee of the present application.

Although much of the previous description is directed at semiconductor wafers, the invention could be applied to any suitable substrate or surface. Further, whenever the terms “automatic,” “automated,” or similar terms are used herein, those terms will be understood to include manual initiation of the automatic or automated process or step. In the following discussion and in the claims, the terms “including” and “comprising” are used in an open-ended fashion, and thus should be interpreted to mean “including, but not limited to . . . ” The term “integrated circuit” refers to a set of electronic components and their interconnections (internal electrical circuit elements, collectively) that are patterned on the surface of a microchip. The term “semiconductor device” refers generically to an integrated circuit (IC), which may be integral to a semiconductor wafer, singulated from a wafer, or packaged for use on a circuit board. The term “FIB” or “focused ion beam” is used herein to refer to any collimated ion beam, including a beam focused by ion optics and shaped ion beams.

To the extent that any term is not specially defined in this specification, the intent is that the term is to be given its plain and ordinary meaning. The accompanying drawings are intended to aid in understanding the present invention and, unless otherwise indicated, are not drawn to scale.

Although the present invention and its advantages have been described in detail, it should be understood that various changes, substitutions and alterations can be made to the embodiments described herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method of preparing a sample for transmission electron microscopy (TEM) analysis, the method comprising:
cleaning the sample to remove a redeposition layer;
imaging the cleaned sample and identifying a location of a region of interest within the sample; and
removing material from the sample, based on the identified location of the region of interest within the sample.

2. The method of claim 1, wherein removing material from the sample comprises:
removing a first portion of the sample using an ion beam oriented in a first direction relative to the sample;
removing a second portion of the sample using an ion beam oriented in a second direction relative to the sample, where the second direction is perpendicular to the first direction.

3. The method of claim 2, wherein cleaning the sample to remove a redeposition layer comprises removing the redeposition layer from a first face of the sample, wherein removing a first portion of the sample exposes a second face of the sample perpendicular to the first face of the sample.

4. The method of claim 3 wherein,
the first portion of the sample comprises a surface of the sample that is angled and/or non-planar, and
wherein removing the first portion of the sample comprises ion milling a portion of the first face of the sample all the way through the sample to expose the second face.

5. The method of claim 3, wherein removing the second portion of the sample comprises removing material from the second face of the sample.

6. The method of claim 5, wherein removing material from the second face of the sample comprises:
applying a fiducial to the second face;
ion milling the second face;
imaging the lamella; and
controlling the ion milling to correct for drift, based on an imaged location of the fiducial.

7. The method of claim 5, wherein removing material from the second face of the sample comprises:
referencing a fiducial adjacent to the second face, where the fiducial is located on a sample grid carrying the sample;
ion milling the second face;
imaging the lamella; and
controlling the ion milling to correct for drift, based on an imaged location of the fiducial.

8. The method of claim 5, wherein removing material from the second face of the sample is based on the identified location of the region of interest within the sample, so that material is removed until a predefined thickness of material remains between the second face of the sample and the region of interest.

9. The method of claim 2, further comprising repositioning the sample between removing the first portion of the sample and removing the second portion of the sample.

10. The method of claim 1, wherein cleaning the sample to remove a redeposition layer comprises removing the redeposition layer from a first face of the sample, wherein imaging the cleaned sample comprises using a charged particle beam to provide an image of the first face of the sample.

11. The method of claim 10, wherein the charged particle beam is an electron beam or an ion beam.

12. The method of claim 1, wherein the sample is a lamella, and wherein the method further comprises separating the lamella from a bulk substrate.

13. The method of claim 12, wherein separating the lamella from the bulk substrate comprises ion beam milling.

14. The method of claim 12, wherein cleaning the sample to remove the redeposition layer is performed during or prior to separating the lamella from the bulk substrate.

15. The method of claim 1, wherein identifying a location of a region of interest within the sample comprises performing image processing techniques to identify an interface between a substrate layer and a structure layer of the sample.

16. The method of claim 15, wherein identifying the location of the region of interest comprises performing image processing techniques via segmentation using a convolutional neural network to reduce a detection area and using image processing methods to identify the interface.

17. An apparatus for preparing a sample for transmission electron microscopy (TEM) analysis, the apparatus comprising:
an ion beam system including an ion beam source, optics for focusing an ion beam along an axis and onto a substrate, and a micromanipulator for manipulating the sample; and
a computing device including a processor and a computer-readable memory storing computer software comprising instructions that, when executed by the processor, cause the apparatus to carry out the method of:
cleaning the sample to remove a redeposition layer;
imaging the cleaned sample and identifying a location of a region of interest within the sample; and
removing material from the sample, based on the identified location of the region of interest within the sample.

18. A non-transitory computer-readable medium storing computing device-executable instructions for preparing a sample for transmission electron microscopy (TEM) analysis, the instructions when executed causing at least one computing device to control an apparatus including an ion beam system to:
clean the sample to remove a redeposition layer;
image the cleaned sample and identify a location of a region of interest within the sample; and
remove material from the sample, based on the identified location of the region of interest within the sample.
